(19) United States
(12) Patent Application Publication
(10) Pub. No.: US 2008/0114287 A1
(43) Pub. Date: May 15, 2008

(54) Ultrasound Microbubble Mediated Genes Delivery System

(76) Inventors: Kar Neng Lai, Hong Kong (HK); Hui Yao Lan, Hong Kong (HK)

Correspondence Address: COOPER & DUNHAM, LLP, 1185 AVENUE OF THE AMERICAS, NEW YORK, NY, 10036, US

(21) Appl. No.: 11/928,109

(22) Filed: Oct. 30, 2007

Related U.S. Application Data
(60) Provisional application No. 60/858,784, filed on Nov. 14, 2006.

(51) Int. Cl. A61M 37/00; A61K 48/00; A61K 9/14

(52) U.S. Cl. 604/23; 514/44; 424/491

(57) ABSTRACT

This invention provides a method for delivery of agents such as genes, plasmids, and other active DNA-related molecules useful for treatment peritoneal disease, including peritoneal fibrosis or postoperative adhesion specifically using an ultrasound-triggered disruption of inducible Smad7 gene-bearing microbubble system.

### FIELD OF THE INVENTION

The current invention relates to a method for peritoneal diseases using a local ultrasound-mediated genes/chemicals-bearing gas-filled microbubble system,

Several publications are referenced herein by Arabic numerals within parenthesis. Full citations for these references may be found at the end of the specification immediately preceding the claims. The references cited herein, including the patents and published patent applications, are incorporated herein by reference.

### BACKGROUND OF THE INVENTION

Although viral-based vectors have been shown to deliver therapeutic DNA effectively into various tissues including cardiovascular system, lung, kidney, and cancers, major concerns remain over the safety using adenovirus and retrovirus, which may elicit immune responses and have the potential for insertional mutagenesis [1, 2]. To overcome these disadvantages, several non-viral approaches have been reported, including lipid-based vector systems and electroporation. Recently, we and other investigators have reported that ultrasound-microbubbles largely increase gene transfection rate in the kidney, cardiovascular tissues, and cancers [3-12]. However, there is no report the use of ultrasound-microbubble-mediated gene/drug treatment for peritoneal diseases including peritoneal fibrosis, postoperative peritoneal adhesion, peritoneal inflammation and cancers.

Ultrasound itself is believed to be harmless to the body and is widely used clinically for many purposes including physical therapy, diagnosis, guidance for deep organ biopsy, and local drug and genetic material delivery, as described in U.S Pat. No. 5,190,766. Most microbubble contrast agents are also safe agents and widely used clinically, as described in U.S. patents, including gas-filled lipids (U.S. Pat. No. 5,580,575), and albumin microbubbles such as Optison [13-16]. Most microbubbles are liquid at room temperature, but they become a gas-filled microbubble with an average of 3 μm in diameter at body temperature. Microbubbles are elastic compressible, and efficient reflectors of ultrasound.

Microbubbles work by resonating in an ultrasound beam, rapidly contracting and expanding in response to the pressure changes of sound waves. Microbubbles can aid drug delivery by themselves, and as agents to carry drugs or genetic materials for site-specific treatment and gene therapy [13-16].

The precise mechanism of gene transfer with ultrasound-microbubble technique remains largely unknown. It may be associated with the effects of sonoporation, the mechanical index and frequency of ultrasound [15, 16]. The principle of the ultrasound-based strategy is that the use of ultrasound contrast agents lowers the threshold for cavitation by ultrasound energy. Using physical properties of microbubbles and coating materials, genes can be incorporated into ultrasound contrast agents [13, 14].

Gene-bearing microbubbles can be injected intravenously or locally and ultrasound energy applied to the target region. As the microbubbles enter the region of insonation, they cavitate and release DNA locally [13, 14]. Cavitation also likely causes a local shockwave that increases cell permeability and thus improves cellular uptake of DNA [13-16]. Scanning electron microscopy also demonstrates that ultrasound with microbubble (Optison) causes a transient formation of holes (<5 μm) in the cell surface, which become undetectable within 24 hours [17].

Peritoneal inflammation/fibrosis is a common disease complication in patients who have end stage renal disease, and who are undergoing maintenance peritoneal dialysis (PD), a most convenient and inexpensive renal replacement therapy. It is a major cause of technical failure of PD, resulting in those PD patients changing to more expensive hemodialysis. In addition, peritoneal adhesions is also a fibrotic process that occurs in a significant proportion of patients undergoing abdominal surgery and contribute to various complications including bowel obstruction, female infertility and chronic abdominal pain, leading to a high morbidity and mortality as well as a high cost of health care. We have shown that the peritoneal fibrosis is mediated by a fibrogenic mediator called transforming growth factor-beta (TGF-β) via its downstream signaling pathway, activation of Smad2/3. The present invention provides ultrasound-triggered gene-bearing microbubbles to locally release an anti-fibrosis and inflammation gene called Smad7 to specifically inhibit the TGF-β/Smad signaling pathway, thereby inhibiting peritoneal fibrosis, as well as peritoneal inflammation, associated with long term peritoneal fibrosis under various disease conditions.

### SUMMARY OF THE INVENTION

The invention provides a method for delivering one or more genes, DNA molecules or plasmids to a patient's peritoneal region for treatment of peritoneal disease therein, comprising providing a source of microbubbles containing one or more genes, DNA molecules, or plasmids for treatment of peritoneal disease; perfusing the peritoneal region of the patient with the microbubbles; providing ultrasonic energy to the abdominal region sufficient to cause transfection of the one or more genes, DNA molecules or plasmids from the microbubbles into the peritoneal region to penetrate peritoneal tissue found therein.

### BRIEF DESCRIPTION OF THE DRAWINGS

Further features and advantages of the invention will become apparent upon review of the following detailed description of the preferred embodiments, taken together with the drawings in which:

FIG. 1 shows photomicrographs and functional data showing the safety of ultrasound microbubble treatment on peritoneal tissues in accordance with the present invention.

FIG. 2 shows the efficiency of ultrasound microbubble mediated Smad7 transgene expression in peritoneal tissues as demonstrated by anti-flag-m2Smad7 immunostaining and RT-PCR (B), where U denotes uremia, PD denotes peritoneal dialysis, and CV denotes control vector.

FIG. 3 demonstrates the mechanism by which ultrasound microbubble mediated Smad7 gene therapy blocks activation, but not expression, of peritoneal TGF-Smad signaling (p-Smad2/3) by Western blot and RT-PCR.

FIG. 4 reveals the therapeutic efficacy of ultrasound-microbubble-mediated Smad7 gene therapy on improving peritoneal functions during the peritoneal dialysis.

FIG. 5 shows photomicrographs of histology and immunohistochemistry, showing that ultrasound-microbubble-mediated Smad7 gene therapy inhibits peritoneal fibrosis associated with peritoneal dialysis in uremia rats by Mason Trichrome (Blue) staining and collagen I immunostaining (Brown).

FIG. 6 is semi-quantitative data showing that ultrasound microbubble mediated Smad7 gene therapy blocks peritoneal fibrosis as determined by inhibiting collagen I and III mRNA and protein expression during peritoneal dialysis.

FIG. 7 is semi-quantitative data showing that ultrasound-microbubble-mediated Smad7 gene therapy inhibits peritoneal fibrosis by blocking α-SMA+myofibroblast transition and fibronectin expression during peritoneal dialysis.

FIG. 8 shows photomicrographs of histology and immunohistochemistry, after ultrasound-microbubble-mediated Smad7 gene therapy. The therapy inhibits peritoneal fibrosis associated with postoperative peritoneal adhesion demonstrated by Mason Trichrome (Blue) staining and collagen I immunostaining (Brown).

FIG. 9 is quantitative real-time PCR data showing that ultrasound-microbubble-mediated Smad7 gene therapy inhibits peritoneal fibrosis associated with postoperative adhesion by blocking collagen I, III, α-SMA, and fibronectin gene expression.

### DETAILED DESCRIPTION OF THE PREFERRED EMBODIMENTS

The invention provides a method for delivering one or more genes, DNA molecules or plasmids to a patient's peritoneal region for treatment of peritoneal disease in the patient, comprising providing a source of microbubbles containing the one or more genes, DNA molecules, or plasmids useful for treating peritoneal disease; perfusing the microbubbles into the peritoneal region of the patient; and administering ultrasonic energy to the peritoneal region sufficient to cause disruption of the one or more genes, DNA molecules or plasmids microbubbles allowing the to penetrate peritoneal tissue found therein.

Preferably, the microbubbles are a plurality of filmogenic protein-encapsulated insoluble microbubbles, and are filled with an insoluble perfluorocarbon gas, such as (without limitation) perfluoromethane, perfluoroethane, perfluoropropane, perfluorobutane, or perfluoropentane. In one embodiment, the microbubbles are about 1 to about 5 microns in diameter.

The ultrasound energy should preferably be administered to the peritoneal region at a frequency of about 0.5-to about 5 MHz, and should be sufficient to cause rupture of the microbubbles within the patient's peritoneal cavity, which includes abdominal wall and mesentery areas. As a person of ordinary skill will appreciate the amount of ultrasonic energy applied should be adjusted so that it is sufficient to degrade, burst, disrupt or break apart the microbubbles without causing damage to peritoneal tissues or the DNA enclosed in the microbubbles. The DNA should be freed from the micrbubbles into adjacent peritoneal tissues, where it can be absorbed by diseased and other cells, and the DNA can incorporate or itself into or transfect the host cell genome.

The peritoneal diseases treatable using the present invention include inflammation, fibrosis, postoperative peritoneal adhesion, or cancer in the peritoneal cavity. The peritoneal disease may be caused by or related to peritoneal infusion/dialysis, surgery, trauma, infection, genetics, or a systemic disease.

Importantly, the one or more DNA molecules, or plasmids include oligonucleotides, DNA, DNA plasmids, siRNA, shiRNA, and micro-RNA. In one embodiment, the DNA molecule is a SMAD7 transgene, while in a preferred embodiment, the DNA molecule is a SMAD7 cDNA.

The present invention thus provides a new strategy for prevention and treatment of peritoneal disease such as peritoneal inflammation/fibrosis and postoperative peritoneal adhesion using the ultrasound-mediated, involving local release of a target gene from the gas-filled microbubbles into the peritoneal tissues. This invention has several advantages.

Ultrasound-microbubble-mediated gene therapy for peritoneal fibrosis is safe because it does not cause either detectable histological and functional damage or cytotoxicity in normal peritoneal tissues. A small DNA molecule with contrast agent is injected into peritoneal cavity followed by transcutaneous ultrasound at the physical therapy levels with 1 MHz at 2 W/cm². Furthermore, it is also feasible to avoid ultrasound-induced heat damage to the tissues by controlling the temperature around 37° C. during ultrasound exposure. We found that energy output at 2 W/cm² with interval exposure time 30 sec for total of 4-6 mins is safe for direct ultrasound exposure to the skin.

An important advantage of the present invention is that ultrasound-mediated gene transfer into the peritoneal tissues is temporary or transient. The transfected Smad7 DNA will be degraded gradually within the peritoneal tissues within 3-4 weeks and repeated gene transfer at day 14 to maintain high levels of Smad7 is feasible. This suggests that, unlike viral-based techniques which mediate a stable transgene expressions ultrasound might not introduce the targeted gene into genome. This may explain why ultrasound mediates a temporary transgene expression. Again, transient expression of Smad7 transgene within the diseased tissues suggests that ultrasound-mediated gene transfer is safer than the virus-based method in terms of the potential for insertional mutagenesis as previously reported [1, 2]. High gene transfection rate is the second advantage of ultrasound microbubble mediated gene transfer. Indeed, we found that more than 80% of peritoneal cells being transfected with Smad7 gene, which is consistent with our previous report that ultrasound substantially increases gene transfection rate in different cell types of normal and disease rat kidney by about 1000-fold [10-12].

Controlling the transfected gene expression within the diseased tissues using the inducible gene therapy as stated in this invention at the therapeutic level without causing side effects is another important advantage of the use of the present invention in gene therapy. For example, while overexpression of Smad7 is able to block TGF-β/Smad signaling and tissue scarring, it is also noted that intensive expression of Smad7 in the kidney induced by higher concentrations of doxycycline results in a massive apoptosis and acute renal injury. Thus, it is critical to control the level of Smad7 transgene expression at the therapeutic level while minimizing side effects when attempting to overexpress Smad7.

Local therapy by direct injection of a mixture of Smad7 gene and microbubble contrast agent into the peritoneal cavity followed by direct ultrasound local treatment via the abdominal skin is a further aspect of the present invention. This largely enhances the local therapeutic effects while it minimizes the side effects due to systemic administration of drugs or genes through an intravenous route.

Ultrasound mediated gene transfer into the peritoneal tissues from the gene-bearing microbubbles locally is a safe and effective therapy for peritoneal diseases. Gas filled microbubbles (3 μm in diameter) either in lipid or albumin forms are able to carry drugs/genes to become drug/gene-bearing microbubbles [13-16]. Microbubbles are elastic, compressible, and efficient reflectors of ultrasound. Microbubbles work by resonating in an ultrasound beam, rapidly contracting and expanding in response to the pressure changes of the sound wave, resulting in lowering of the threshold for cavitation by ultrasound energy [13-16]. Gene-bearing microbubbles can be injected intravenously or locally and ultrasound energy applied to the target region. As the microbubbles enter the region of insonation, they cavitate, locally releasing DNA and drug materials. Cavitation also likely causes a local shockwave that increases cell permeability and thus improves cellular uptake of DNA [13-16].

In the present study, a mouse Smad7 cDNA with a flag tag (m2) at its NH2 terminus in pcDNA3 was subcloned into a tetracycline-inducible vector, pTRE, to obtain pTRE-m2Smad7. To achieve doxycycline (a tetracycline derivative)-induced Smad7 transgene expression, pTRE-m2Smad7 and an improved pTet-on vector, pEFpurop-Tet-on were co-transfected into the peritoneal cavity. In the pilot study, we found that the peak of gene expression of exogenous Smad7in peritoneal tissues was observed on the second day of transfection and the expression of transgene decreased in a time-dependent manner. To ensure the effectiveness of transfection, exogenous Smad7 was administered into peritoneal cavity on the first day and 14^{th} day of PD in uremic rats. The uremic PD rats receiving empty vectors without Smad7 insert were taken as the treatment controls. The procedures of transfection were as follows. The rats were anesthetized by inhalation of isoflurane. The mixture of plasmids and microbubbles (Optison, Amersham Health Inc., Princeton, N.J., USA or SonoVue, Bracco International B.V., Amsterdam, Netherlands) was prepared with 1:1 vol/vol ratio. Then 4 ml of the mixed solution containing 100 μg of plasmids were immediately injected into the abdominal cavity. Ultrasound mediating gel was then applied on the shaved abdominal skin. The ultrasound transducer (Sonitron 2000, Rich-Mar Corp., Inola, Oklah., USA) was applied directly onto the abdominal wall with 1-MHz input frequency, 2 W/cm² output intensity and 20% duty cycle for a total of 4 mins with 30 s intervals. After the gene transfer procedures, one milliliter of doxycycline (500 μg/ml, Sigma) was injected into the peritoneal cavity to induce Smad7 transgene expression, followed by 200 μg/ml of doxycycline in drinking water to maintain the induction of transgene expression.

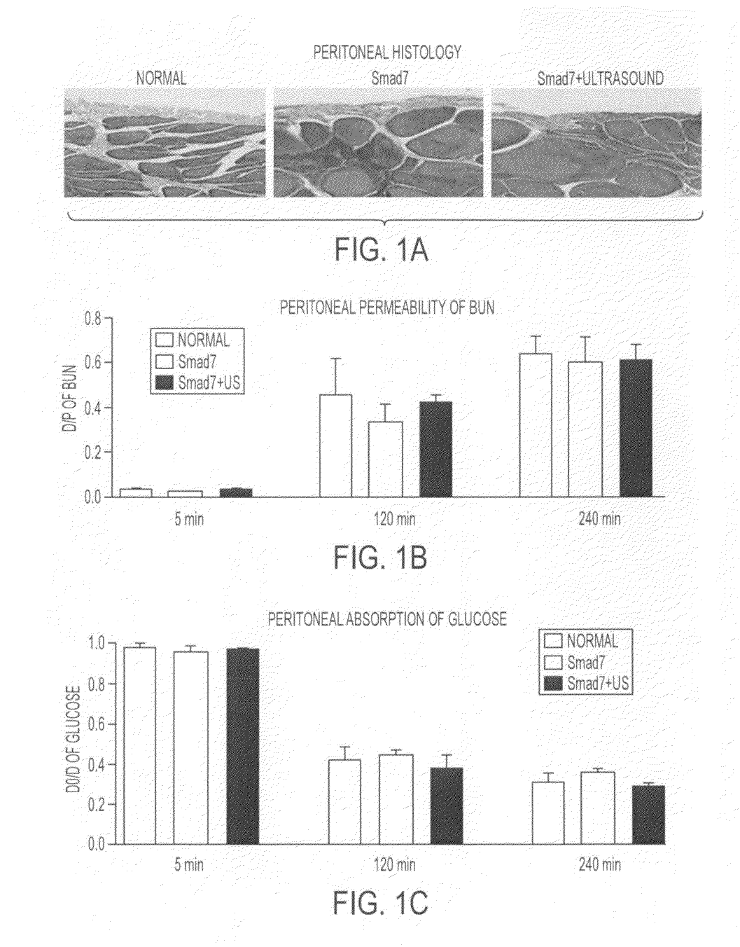

Safety with ultrasound microbubble mediated gene therapy for peritoneal fibrosis is the most important aspect of this invention. Ultrasound microbubble mediated gene therapy for peritoneal fibrosis is safe because it is noninvasive and does not cause either detectable histological and functional damage or cytotoxicity in normal peritoneal tissues by injecting a small DNA with contrast agent into peritoneal cavity followed by 4 minutes transcutaneous ultrasound at the physical therapy levels with 1 MHz at 2 W/cm². Ultrasound itself is harmless to the body and is widely used clinically for many purposes including physical therapy, diagnosis, guidance for deep organ biopsy, and local drug and genetic material delivery [13-16]. The microbubble contrast agents are also safe and widely used clinically. It has been well documented that microbubbles can aid drug delivery by themselves and as agents to carry drugs or genetic materials for site specific treatment and gene therapy [13-16]. In addition, injection of small DNA into the peritoneal cavity is also safe. It is also feasible to avoid ultrasound-induced heat damage to the tissues by controlling the temperature around 37° C. during ultrasound exposure. We found that energy output at 2 W/cm² with interval exposure time 30 sec for up to 6 mins is safe for direct ultrasound exposure to the skin. As demonstrated in FIG. 1, there is no detectable histological or functional damage associated with ultrasound microbubble treatment. Most importantly, we also found that ultrasound mediated gene transfer into the peritoneal tissues is temporary, which mimics the clinical therapies. The transfected Smad7 DNA will be degraded gradually within the peritoneal tissues within 3-4 weeks and repeated gene transfer at day 14 to maintain high levels of Smad7 is feasible since it is practical and no invasive. This suggests that, unlike viral-based techniques which mediate a stable transgene expression, ultrasound might not introduce the targeted gene into the genome. This may explain why ultrasound mediates a temporary transgene expression. Again, transient expression of Smad7 transgene within the diseased tissues suggests that ultrasound-mediated gene transfer is safer than the virus-based method in terms of the potential for insertional mutagenesis [1,2]. Taken together, the ultrasound microbubble gene therapy method is safe.

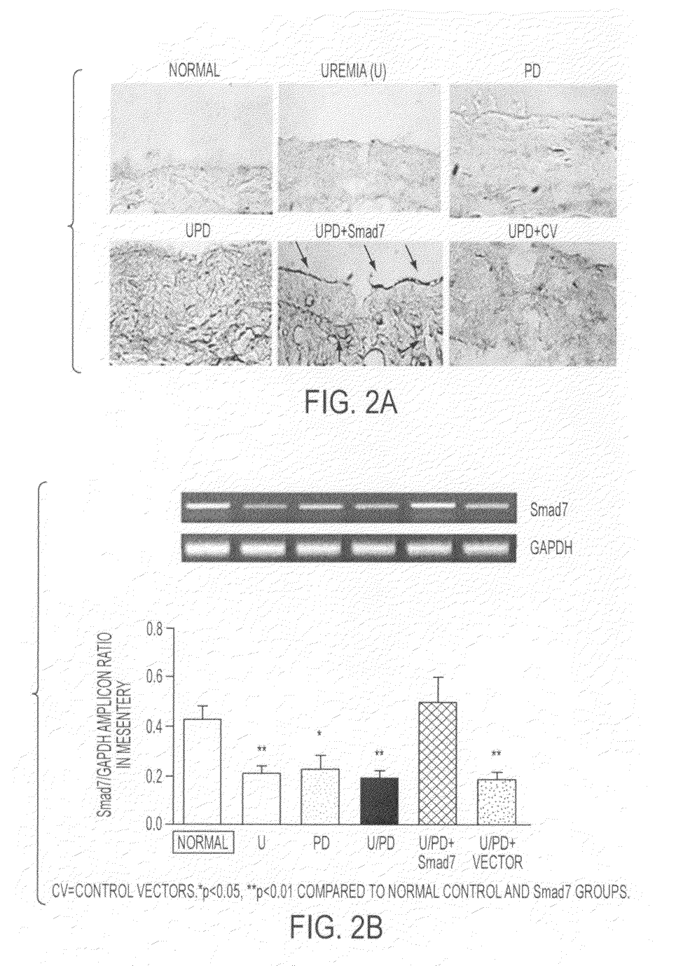

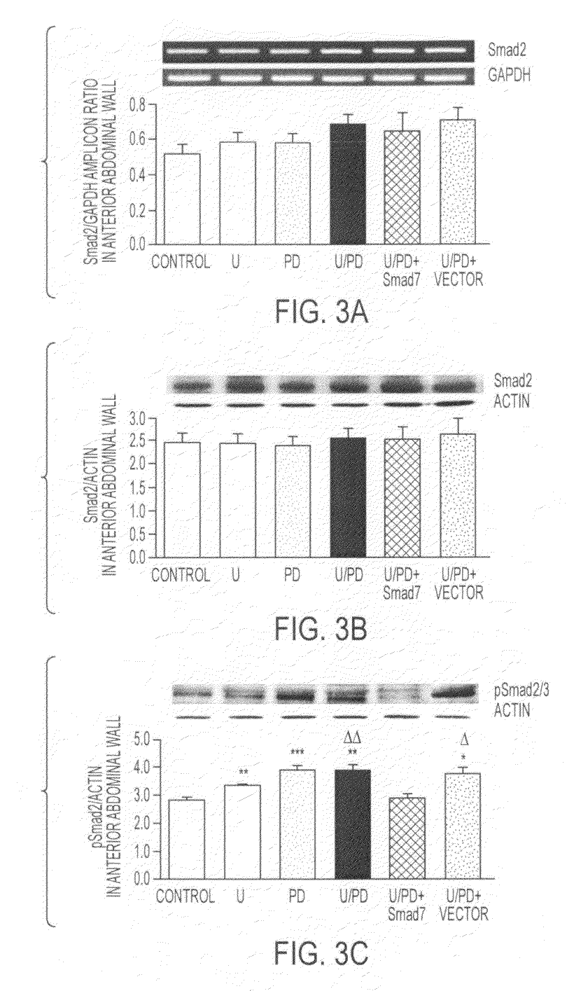

High gene transfection rate mediated by ultrasound-microbubble system is another distinct advantage of this invention. It has long been noted that low gene transfection rate is a major disadvantage when using non-viral-based gene delivery systems such as naked DNA and liposome. Using ultrasound-triggered gene released from microbubbles, a high gene transfection rate was evident by the finding that more than 80% of total peritoneal cells on the surface mesothelial cell layer and submesothelial cells are positive for flag-M2 Smad7 transgene (FIG. 2A), resulting in a marked upregulation of Smad7 (FIG. 2B), which is a key mechanism of anti-peritoneal fibrosis since overexpression of Smad7 is able to block activation of TGF-β/Smad signaling by inhibiting Smad2/3 phosphorylation (FIG. 3C).

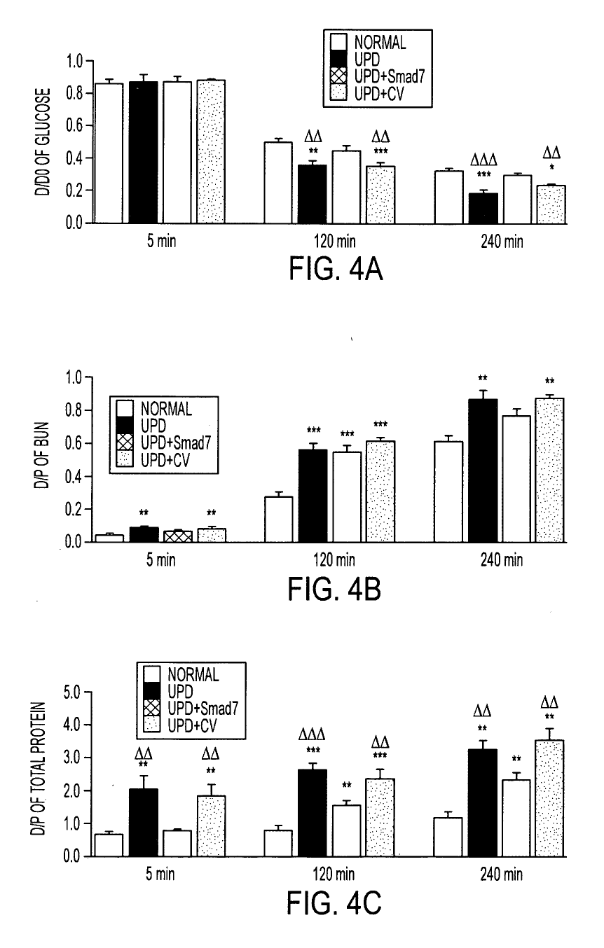

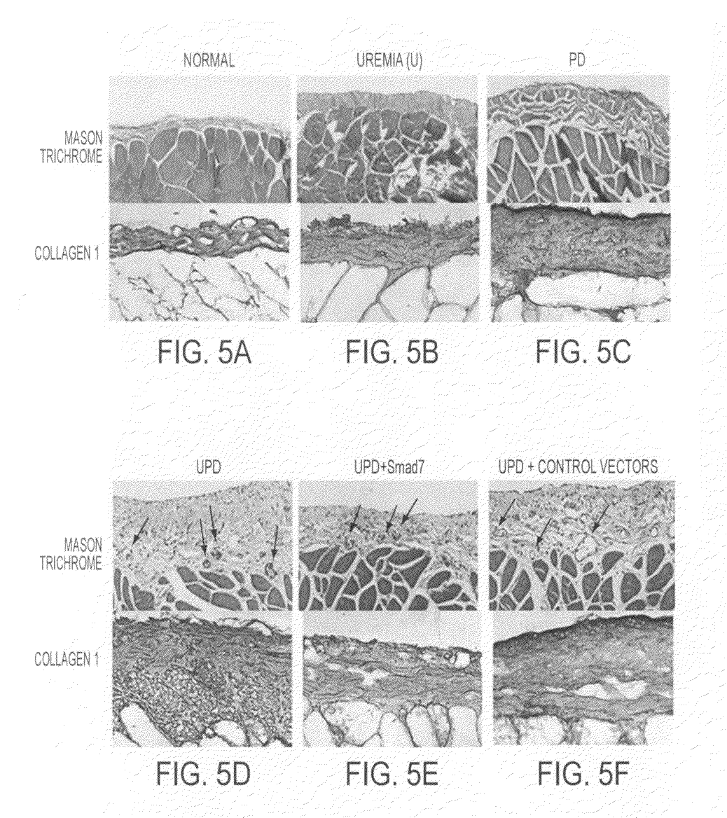

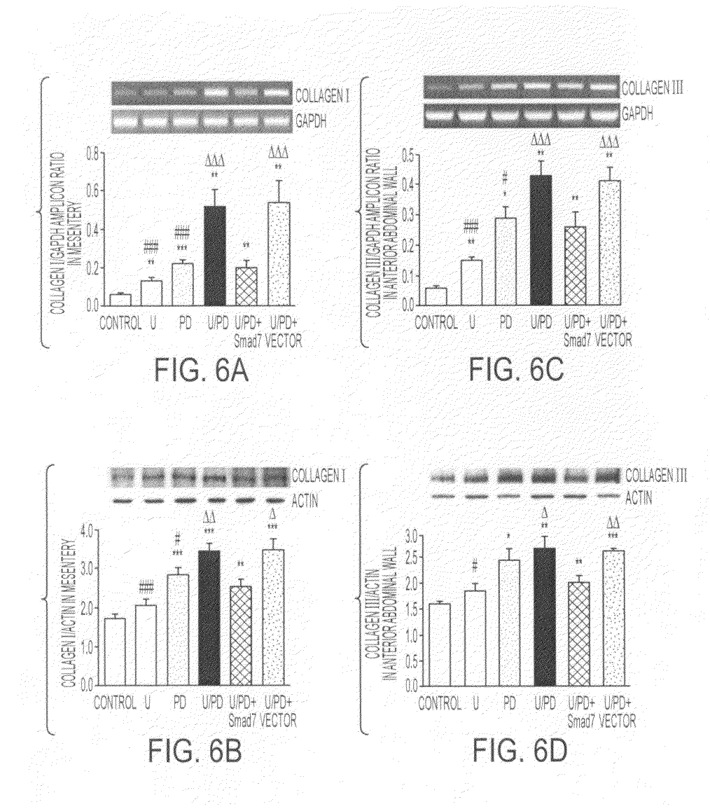

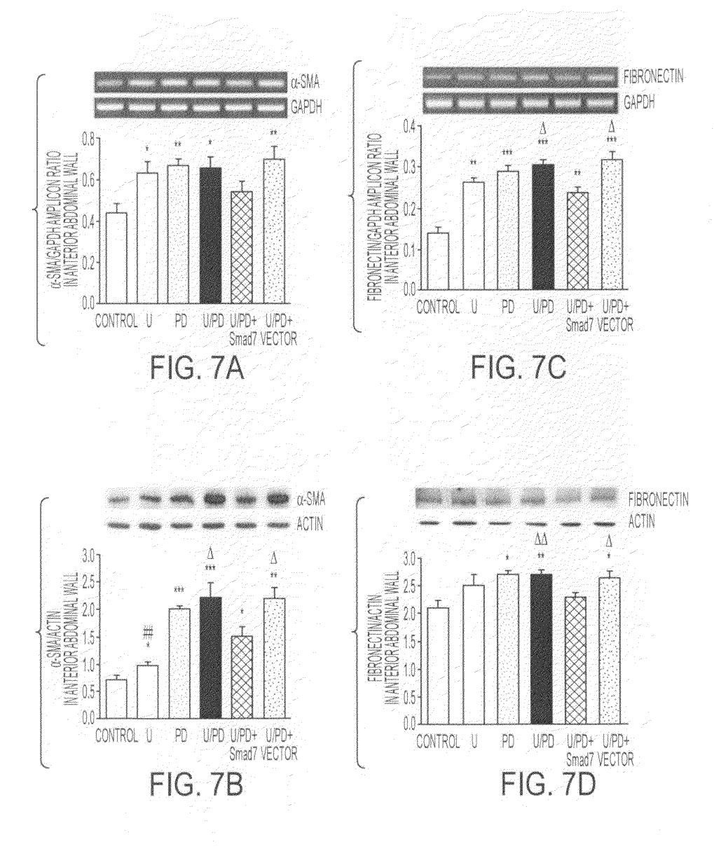

The effectiveness of prevention and treatment of peritoneal fibrosis using ultrasound microbubble technique is a key aspect of this invention. Although peritoneal fibrosis is a major cause and common feature of technique failure of peritoneal dialysis, it is not yet available for specific and effective therapies for preventing and treating this disease. This invention has shown that ultrasound mediated release of Smad7 gene from albumin-type microbubbles is able to substantially inhibit the development of peritoneal fibrosis associated with peritoneal dialysis in uremia rats that have clinical features of end stage renal disease. As demonstrated in FIGS. 1-7, overexpression of peritoneal Smad7 by ultrasound-microbubble-mediated gene therapy results in substantial inhibition of Smad2/3 activation (FIG. 3C), thereby blocking peritoneal dialysis related peritoneal fibrosis, as illustrated by preserving peritoneal function (FIG. 4), attenuating peritoneal fibrotic thickening (FIG. 5), and preventing peritoneal fibrosis through inhibition of collagen I, III, fibronectin, and α-SMA expression (FIGS. 6, 7).

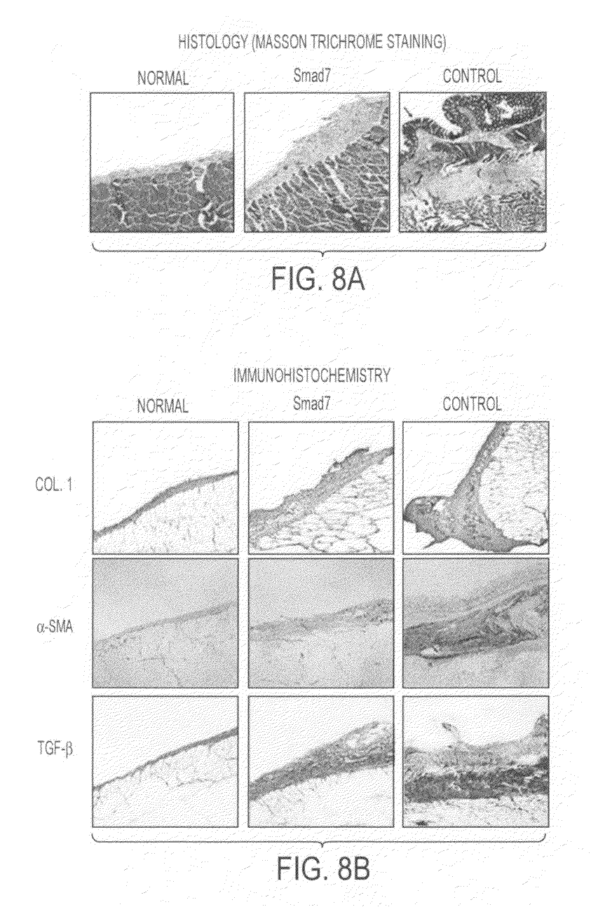

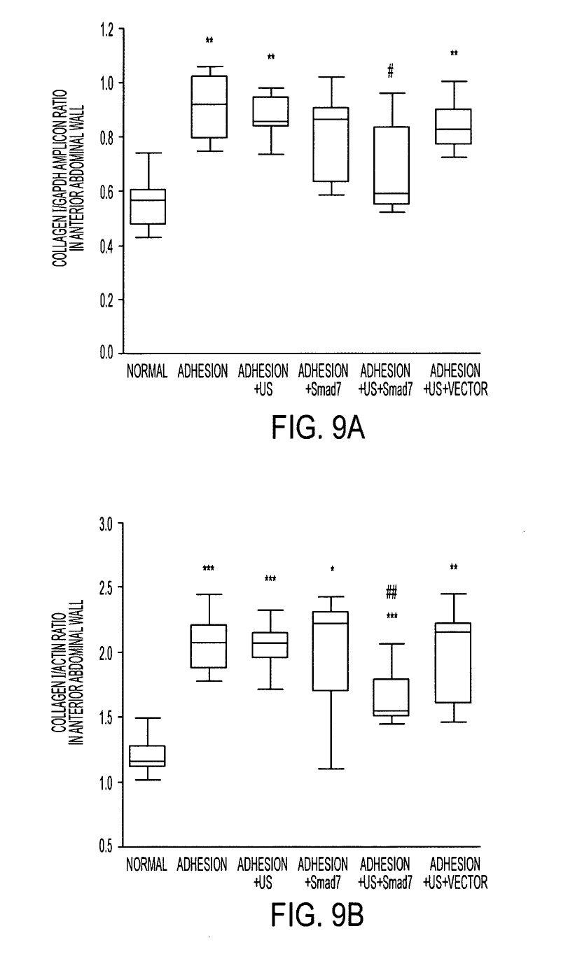

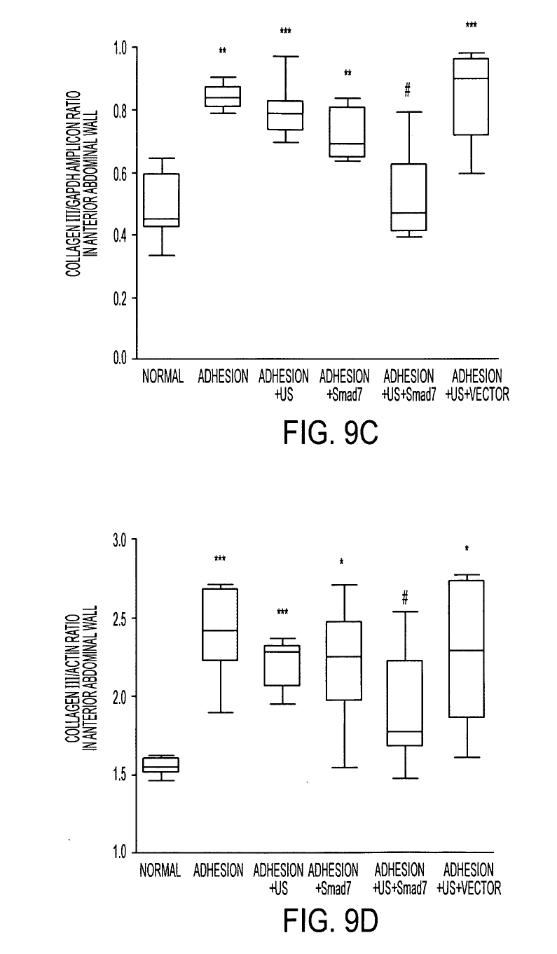

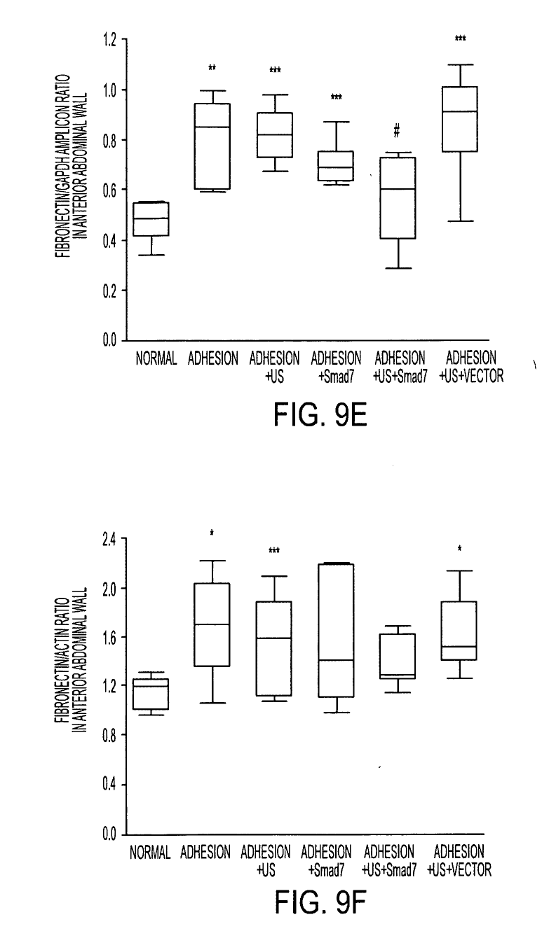

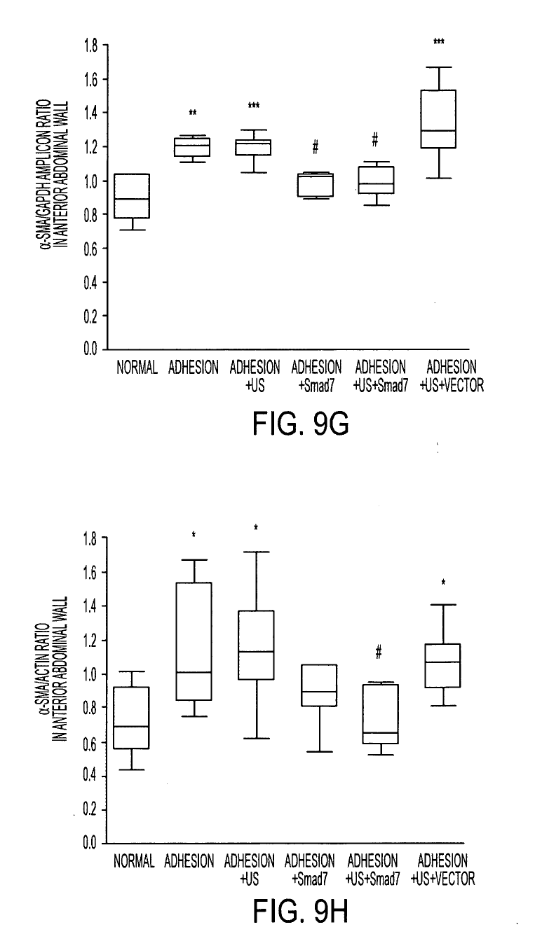

In addition, this invention also demonstrates that ultrasound-mediated gene expression of Smad7 is able to block peritoneal fibrosis associated with the postoperative peritoneal adhesion/fibrosis in rats (FIG. 8). Indeed, after four weeks of surgical abrasion, the rats developed significant peritoneal adhesions including overexpression of α-SMA, collagen I, III, and fibronectin. The enhanced expression of TGF-β and activation of TGF-β/Smad signaling are blocked by ultrasound-microbubble-mediated Smad7 transfection (FIGS. 8, 9). Thus, blockade of TGF-β/Smad signaling pathway via an ultrasound-microbubble-mediated system represents a safe and novel therapeutic approach for preventing postsurgical peritoneal adhesions.

Controlling the transfected gene expression within the diseased tissues using inducible gene therapy, as set forth herein, at the therapeutic level but without causing side effects is another important aspect of the present invention, and has application to gene therapy. For example, while overexpression of Smad7 is able to block TGF-β/Smad signaling and tissue scarring, our previous finding also shows that intensive expression of Smad7 in the kidney induced by higher concentrations of doxycycline results in a massive apoptosis and acute renal injury [10]. Thus, it is critical to control the level of Smad7 transgene expression at the therapeutic level while minimizing side effects when attempting to overexpress Smad7.

Local therapy using the direct peritoneal injection of Smad7-bearing microbubbles followed by local ultrasound application over the abdominal skin is the most appreciative one. This avoids the use of systemic treatment. Indeed, traditional treatment for peritoneal diseases relies on the systemic administration of drugs. This approach is usually not effective and may also cause undesirable side effects. The invention has overcome this disadvantage and largely improved the effectiveness of local expression of negative TGF-β signaling molecule Smad7, resulting in inhibition of TGF-β/Smad-mediated peritoneal fibrosis.

The following references are incorporated by reference herein:
1. Check E: Gene therapy: a tragic setback. Nature 2002; 420:116-8.
2. Haviernik P, Bunting K D: Safety concerns related to hematopoietic stem cell gene transfer using retroviral vectors, Curr. Gene Ther. 2004; 4:263-76.
3. Bekeredjian R, Chen S, Frenkel P A, Graburn P A, Shohet R V. Ultrasound-targeted microbubble destruction can repeatedly direct highly specific plasmid expression to the heart. Circulation 2003; 108:1022-26.
4. Korpanty G, Chen S, Shohet R V et al. Targeting of VEGF-mediated angiogenesis to rat myocardium using ultrasonic destruction of microbubbles. Gene Ther. 2005; 12:1305-12.
5. Lu Q L, Liang H D, Partridge T, Blomley M J. Microbubble ultrasound improves the efficiency of gene transduction in skeletal muscle in vivo with reduced tissue damage. Gene Ther. 2003; 10:396-405.
6. Taniyama Y, Tachibana K, Hiraoka K et al. Local delivery of plasmid DNA into rat carotid artery using ultrasound. Circulation 2002; 105:1233-39.
7. Shimamura M, Sato N, Taniyama Y et al. Development of efficient plasmid DNA transfer into adult rat central nervous system using microbubble-enhanced ultrasound. Gene Ther. 2004; 11:1532-39.
8. Manome Y, Nakamura M, Ohno T, Furuhata H. Ultrasound facilitates transduction of naked plasmid DNA into colon carcinoma cells in vitro and in vivo. Hum. Gene Ther. 2000; 11:1521-28.
9. Sakakima Y, Hayashi S, Yagi Y, Hayakawa A, Tachibana K, Nakao A. Gene therapy for hepatocellular carcinoma using sonoporation enhanced by contrast agents. Cancer Gene Ther. 2005; 12:884-9.
10. Lan H Y, Mu W, Tomita N et al. Inhibition of renal fibrosis by gene transfer of inducible Smad7 using ultrasound-microbubble system in rat UUO model. J. Am. Soc. Nephrol. 2003; 14:1535-48.
11. Moullier P, Friedlander G F, Calise D, Ronco P, Perricaudet M, Ferry N: Adenoviral-mediated gene transfer to renal tubular cells in vivo. Kidney Int 45:1220-1225, 1994.
12. Imai E, Isaka Y: Strategies of gene transfer to the kidney. Kidney Int 53: 264-272, 1998.
13. Tomita N, Morishita R, Lan H Y et al: In vivo administration of a nuclear transcription factor-κB decoy suppresses experimental crescentic glomerulonephritis. J Am Soc Nephrol 11: 1244-1252, 2000.
14. Tsujie M, Isaka Y, Nakamura H, Imai E, Hori M: Electroporation-mediated gene transfer that targets glomeruli. J Am Soc Nephrol 12: 949-954, 2002.
15. Hou C C, Wang W, Huang X R et al. Ultrasound-microbubble-mediated gene transfer of inducible Smad7 blocks transforming growth factor-beta signaling and fibrosis in rat remnant kidney. Am. J. Pathol. 2005; 166:761-71.
16. Ng Y Y, Hou C C, Wang W, Huang X R, Lan H Y. Blockade of NF-kappaB activation and renal inflammation by ultrasound-mediated gene transfer of Smad7 in rat remnant kidney. Kidney Int. 2005; 94:S83-91.
17. Bekeredjian R, Grayburn P A, Shohet R V. Use of ultrasound contrast agents for gene or drug delivery in cardiovascular medicine. J. Am. Coll. Cardiol. 2005; 45:329-35.
18. Unger E C, Matsunaga T O, McCreery T, Schumann P, Sweitzer R, Quigley R. Therapeutic applications of microbubbles. Eur. J. Radiol. 2002, 42:160-8.
19. Blomley M J K, Cooke J C, Unger E C, Monaghan M J, Cosgrove D O: Science, medicine, and the future: Microbubble contrast agents: a new era in ultrasound. BMJ 322: 1222-1225, 2001.
20. Harvey C J, Blomley M J, Eckersley R J, Cosgrove D O: Developments in ultrasound contrast media. Eur Radiol 11: 675-689, 2001.
21. Taniyama Y, Tachibana K, Hiraoka K, Namba T, Yamasaki K, Hashiya N, et al. Local delivery of plasmid DNA into rat carotid artery using ultrasound. Circulation 2002; 105:1233-1239.

What is claimed is:

1. A method for delivering one or more genes, DNA molecules or plasmids useful for treatment of a peritoneal disease to a patient's peritoneal region for treatment of a peritoneal disease therein, comprising:
providing a source of microbubbles containing the one or more genes, DNA molecules, or plasmids for treating peritoneal disease;
perfusing the peritoneal region of the patient with the microbubbles; and
administering ultrasonic energy to the abdominal or peritoneal region sufficient to cause transfection of the one or more genes, DNA molecules or plasmids into the abdominal or peritoneal tissue of the patient.

2. The method according to claim 1, wherein the microbubbles comprise a plurality of filmogenic protein-encapsulated insoluble microbubbles.

3. The method of claim 1, wherein the microbubbles are filled with an insoluble perfluorocarbon gas.

4. The method of claim 3, wherein the perfluorocarbon gas is perfluoromethane, perfluoroethane, perfluoropropane, perfluorobutane, or perfluoropentane.

5. The method of claim 1, wherein the microbubbles are 1 to 5 microns in diameter.

6. The method according to claim 3, wherein the ultrasound energy is administered at a frequency of about 0.5 to about 5 MHz.

7. The method of claim 6, wherein the ultrasound energy causes disruption of the microbubbles in the peritoneal tissues.

8. The method according to claim 1, wherein the peritoneal tissues include those tissues within the patient's peritoneal cavit
